(19) United States
(12) Patent Application Publication
(10) Pub. No.: US 2018/0235510 A1
(43) Pub. Date: Aug. 23, 2018

(54) SIMPLIFIED DISPLAY OF END-TIDAL CO2

(71) Applicant: KONINKLIJKE PHILIPS N.V., Eindhoven (NL)

(72) Inventors: Joseph Allen ORR, Park City, UT (US); Lara Marie BREWER CATES, Bountiful, UT (US)

(21) Appl. No.: 15/751,253

(22) PCT Filed: Aug. 4, 2016
(86) PCT No.: PCT/IB2016/054702

§ 371 Date: Feb. 8, 2018

Related U.S. Application Data
(60) Provisional application No. 62/203,090, filed on Aug. 10, 2015.

(51) Int. Cl. A61B 5/083; A61B 5/00

(52) U.S. Cl. CPC A61B 5/0836; A61B 5/082; A61M 2230/432; A61B 5/725

(57) ABSTRACT

A capnograph device includes a carbon dioxide measurement component (20) configured to measure respiratory carbon dioxide level, and an electronic processor (30) programmed to generate a capnogram signal (40) and compute an end-tidal carbon dioxide (etCO₂) signal (50) by performing a sliding window maximum operation (42, 44) on the capnograph signal. In some embodiments the sliding window maximum operation employs a sliding time window (W) whose duration (T_{w}) is at least 30 seconds. A smoothing filter may be applied to the capnograph signal before performing the sliding window maximum operation, and/or a smoothing filter (52) may be applied after the sliding window maximum operation to produce a smoothed etCO₂ signal (54). The capnograph device may be a sidestream capnograph device (10) or a mainstream capnograph device.

### FIELD

The following relates generally to the capnography arts and related arts.

### BACKGROUND

A capnography device monitors the concentration or partial pressure of carbon dioxide (CO₂) in respiratory gases. A common capnography parameter is the end-tidal CO₂ (etCO₂) which conceptually is the CO₂ partial pressure at the end of the exhalation phase. However, since this is usually the largest observed CO₂ partial pressure in the breathing cycle, etCO₂ is clinically defined as the maximum observed CO₂ partial pressure over the breathing cycle. The etCO₂ is commonly presented as a partial pressure (PetCO₂) or as a percentage value.

The etCO₂ parameter measured by capnography is commonly employed as a measurable surrogate for the maximum carbon dioxide partial pressure at the alveoli of the lungs. Knowledge of the maximum alveolar CO₂ partial pressure, in turn, is useful for diagnosing the state of the pulmonary and cardiopulmonary systems, and accordingly has substantial value for clinical diagnosis and patient monitoring. A stable etCO₂ trend line indicates stable respiration, while if the etCO₂ is trending downward over time this can indicate respiratory deterioration, adverse reaction to medication, impact of anesthesia or sedation, or so forth.

However, the etCO₂ measured by capnography is often noisy, and can vary significantly from breath to breath. The capnography etCO₂ can vary with changes in breathing pattern, when the patient engages in talking, coughs, or so forth.

The following discloses a new and improved systems and methods that address the above referenced issues, and others.

### SUMMARY

In one disclosed aspect, a capnograph device is disclosed, including a carbon dioxide measurement component configured to measure respiratory carbon dioxide level and an electronic processor programmed to: generate a capnogram signal comprising respiratory carbon dioxide level measured by the carbon dioxide measurement component as a function of time; and compute an end-tidal carbon dioxide (etCO₂) signal as a function of time by operations including performing a sliding window maximum operation on the capnograph signal. In some embodiments the sliding window maximum operation employs a sliding time window whose duration is at least 30 seconds. In some embodiments performing the sliding window maximum operation comprises computing etCO₂(t)=max([CO₂])|_{W(t)} where t denotes time, [CO₂] is the capnogram signal (40) and W(t) is a sliding time window. The capnograph device may be a sidestream or mainstream capnograph device.

In another disclosed aspect, a non-transitory storage medium stores instructions readable and executable by an electronic processor to perform a capnography method comprising: generating a capnogram signal comprising respiratory carbon dioxide level measured by a carbon dioxide measurement component as a function of time; and performing a sliding window maximum operation on the capnograph signal to compute an end-tidal carbon dioxide (etCO₂) signal as a function of time.

One advantage resides in providing an end-tidal carbon dioxide (etCO₂) value that more accurately approximates the maximum alveolar carbon dioxide level.

Another advantage resides in providing etCO₂ with reduced noise compared with end-tidal CO₂ determined on a breath-by-breath basis.

Another advantage resides in providing etCO₂ that both (1) more accurately approximates the maximum alveolar carbon dioxide level and (2) has reduced noise compared with end-tidal CO₂ determined on a breath-by-breath basis.

Another advantage resides in providing etCO₂ with reduced systematic error.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a capnograph device including improved end-tidal carbon dioxide (etCO₂) calculation as disclosed herein.

FIG. 2 diagrammatically illustrates performing a sliding window maximum operation on a capnogram to compute etCO₂.

FIGS. 3 and 4 plot end-tidal CO₂ data computed on a breath-by-breath basis (FIG. 3) and by using a sliding window maximum operation (FIG. 4).

### DETAILED DESCRIPTION

The trend of etCO₂ is difficult to evaluate when the patient is spontaneously breathing and the breaths are not uniform in size. During spontaneous or pressure supported ventilation, the etCO₂ as measured by a capnograph device can vary significantly, for example when the patient talks, coughs, suffers from sleep apnea or drug induced airway obstruction or experiences acute respiratory depression after anesthesia for a medical procedure. It is not physiologically possible for the alveolar CO₂ partial pressure to change as quickly as the etCO₂ changes observed by capnography with breaths of varying size.

An apparent solution is to smooth the etCO₂ trend line using a low pass filter or the like to remove the noise. However, it is recognized herein that this approach has significant disadvantages in the case of etCO₂ measured by capnography. This is because, as recognized herein, clinical conditions and physiological events that introduce noise into the etCO₂ measurement tend to systematically reduce the etCO₂ as measured by the capnograph device. For example, if the volume of the breath is too small to completely flush out the airway dead volume, the measured etCO₂ will be reduced. Similarly, if the lungs contain parallel (alveolar) dead volume, the etCO₂ measured by capnography will again be reduced. If supplemental oxygen is being administered to the patient, it can combine with the exhaled gas and, yet again, reduce the etCO₂ reading produced by capnography.

A common clinical application of etCO₂ measurement by capnography is to provide an accurate, measurable surrogate for the maximum alveolar CO₂ partial pressure which is not directly measurable. However, each of the foregoing etCO₂ noise sources causes a reduction in the etCO₂ value measured by capnography, so as to systematically deviate below the alveolar maximum CO₂ partial pressure. When the etCO₂ measured by capnography is viewed as a surrogate for the alveolar maximum CO₂ partial pressure, these “noise” sources are therefore not true noise sources that introduce random error. Rather, these “noise” sources are sources of systematic error, in that they systematically cause the etCO₂ measured by capnography to read too low when compared with the (not readily measured) gold standard of the alveolar maximum CO₂ partial pressure.

When viewed in light of the foregoing insights, a low pass filter or other smoothing mechanism designed to remove noise, i.e. random error, is not appropriate for improving the etCO₂ values measured by capnography. Rather, the appropriate improvement should preferentially display the maximum observed CO₂ over a relatively long period of time (e.g. encompassing around 10-30 breaths), as this is more likely to present etCO₂ values that accurately reflect the maximum alveolar CO₂. In some illustrative embodiments, the following processing is disclosed. At a fixed sampling time interval T_{S}, e.g. 5-15 seconds in some embodiments, the maximum expired CO₂ measured over a time window W of longer interval T_{W}, e.g. 30 seconds-to-3 minutes in some embodiments, and 1-2 minutes in some embodiments, is identified. These maximum samples obtained at the sampling rate (1/T_{S}) form a sampled signal representing the etCO₂, with successive data points (samples) of the signal spaced apart by the sampling interval T_{S}. Optionally, this etCO₂ signal is smoothed, for example using a low-pass filter, to remove spurious samples (these are true noise, i.e. are expected to constitute random error).

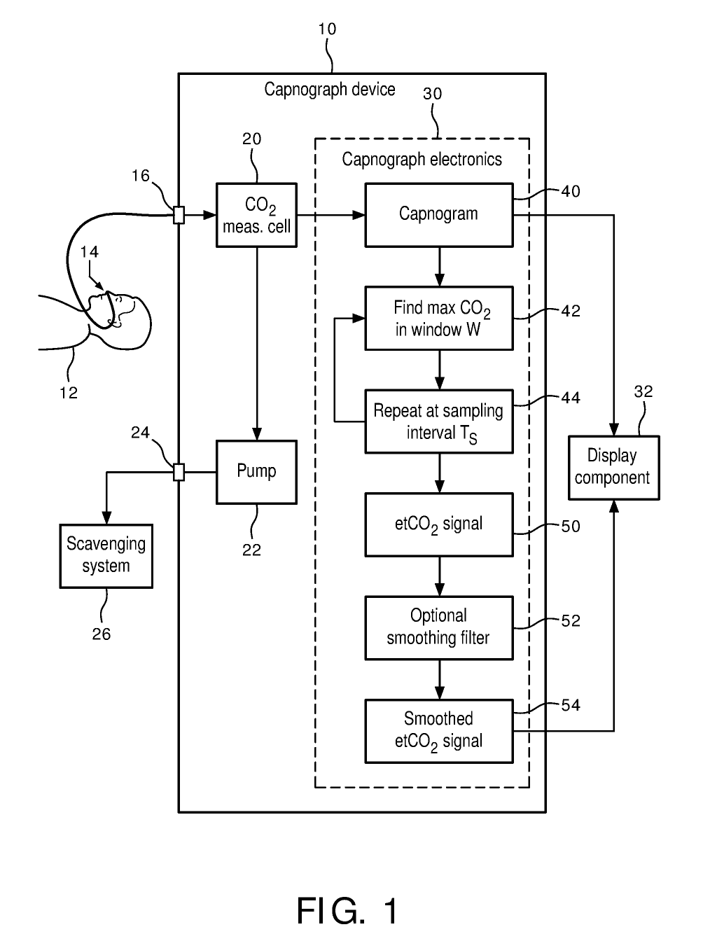

With reference to FIG. 1, an illustrative capnograph device 10 employing such etCO₂ signal generation is diagrammatically shown. As shown in FIG. 1, during operation the capnograph device 10 is connected with a patient 12 by a suitable patient accessory, such as a nasal cannula 14 in the illustrative example, or by an airway adaptor or so forth. The patient accessory 14 may optionally include one or more ancillary components, such as an air filter, water trap, or the like (not shown). In the illustrative capnograph 10, respired air is drawn from the patient accessory 14 into a capnograph air inlet 16 and through a carbon dioxide (CO₂) measurement component or cell 20 by an air pump 22. The air is then discharged via an air outlet 24 of the capnograph 10 to atmosphere or, as in the illustrative embodiment, is discharged through the air outlet 24 into a scavenging system 26 to remove an inhaled anesthetic or other inhaled medicinal agent before discharge into the atmosphere. The CO₂ measurement component or cell 20 may, for example comprise an infrared optical absorption cell in which carbon dioxide in the respired air drawn from the patient accessory 14 produces absorption that is detected by an infrared light source/detector assembly.

The illustrative capnograph device 10 has a sidestream configuration in which respired air is drawn into the capnograph device 10 using the pump 22, and the CO₂ measurement cell 20 is located inside the capnograph device 10. That is, the sidestream capnograph device 10 includes, as a unit, the carbon dioxide measurement component 20, the electronic processor 30, and the pump 22 connected to draw respired air though the carbon dioxide measurement component 20. The sidestream configuration is suitably used for a spontaneously breathing patient, i.e. a patient who is breathing on his or her own without assistance of a mechanical ventilator. In an alternative configuration, known as a mainstream configuration (not illustrated), the CO₂ measurement cell is located externally from the capnograph device housing, typically as a CO₂ measurement cell patient accessory that is inserted into the “mainstream” airway flow of the patient. Such a mainstream configuration may, for example, be employed in conjunction with a mechanically ventilated patient in which the CO₂ measurement cell patient accessory is designed to mate into an accessory receptacle of the ventilator unit, or is installed on an airway hose feeding into the ventilator. The disclosed approaches for calculating etCO₂ are readily applied either in conjunction with a sidestream capnograph device (as in the illustrative example of FIG. 1) or in conjunction with a mainstream capnograph device.

With continuing reference to FIG. 1, the capnograph device 10 (in either the illustrative sidestream configuration or in the alternative mainstream configuration) includes capnograph electronics 30 which provide power and control for operating the CO₂ measurement cell 20 and (in the sidestream configuration) the pump 22. Note that the power and control links are not illustrated in diagrammatic FIG. 1. The capnograph electronics 30 additionally perform processing of the CO₂ signal output by the CO₂ measurement cell 20, as diagrammatically indicated in FIG. 1 and as described herein. Clinical data output by the capnograph 10, such as a capnogram and etCO₂ signal, are displayed on a display component 32, stored in an electronic medical record (EMR) or the like, or otherwise utilized. The display component 32 may be a component of the capnograph or, as illustrated in FIG. 1, the display component 32 may be an external display component connected to the capnograph 10. For example, the external display component 32 may be a multi-function bedside patient monitor and/or a nurses' station patient monitor or so forth. It will be further appreciated that the capnograph may include numerous other components not illustrated in simplified diagrammatic FIG. 1, such as a pressure gauge, flow meter, and so forth.

The capnograph electronics 30 may be variously implemented, such as by a suitably programmed electronic processor, e.g. a microprocessor or microcontroller of the capnograph 10. While a single electronics unit 30 is illustrated, it is alternatively contemplated to employ various combinations of electronics, for example different electronic components may be operatively interconnected to implement a pump power supply, infrared light source power supply (for the CO₂ measurement cell 20), analog-to-digital conversion circuitry (to sample the infrared light detector of the CO₂ measurement cell 20), and so forth. Still further, it is contemplated for the capnograph to output the capnogram (CO₂ versus time signal) without the disclosed CO₂ signal processing and for that processing to be performed by suitably programmed electronics in another device (for example, the computer of a nurses' station that receives the capnogram signal). It will be still further appreciated that the CO₂ signal processing disclosed herein as being performed by the capnograph electronics 30 may be embodied by a non-transitory storage medium storing instructions that are readable and executable by the microprocessor, microcontroller, or other electronic processor to perform the disclosed CO₂ signal processing including the etCO₂ calculation employing approaches disclosed herein. Such non-transitory storage media may, by way of non-limiting illustration, include a hard disk drive or other magnetic storage medium, a flash memory, read-only memory (ROM) or other electronic storage medium, an optical disk or other optical storage medium, various combinations thereof, or so forth.

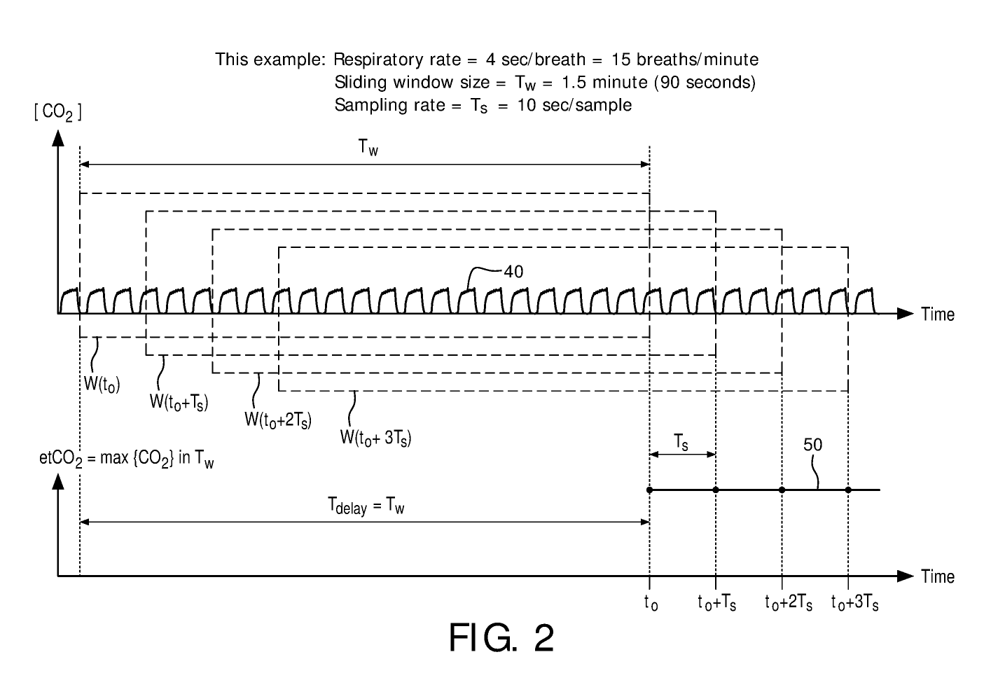

With continuing reference to FIG. 1 and with further reference to FIG. 2, an illustrative embodiment of the CO₂ signal processing performed by the capnograph electronics 30 (or alternatively in whole or in part by a nurses' station monitor, bedside patient monitor, or other device with a suitably programmed electronic data processor) is diagrammatically shown in FIG. 1. The CO₂ signal is sampled and optionally corrected for factors such as the presence of interfering gases (e.g. nitrous oxide), barometric pressure, and so forth in order to generate a capnogram 40. The capnogram is a signal representing the partial pressure or concentration of carbon dioxide, denoted in FIG. 2 as [CO₂], as a function of time. Diagrammatic FIG. 2 illustrates the capnogram 40 as an idealized waveform for a healthy patient, in which every breath is identical and exhibits near-zero [CO₂] during the inspiratory phase and a well-defined maximum [CO₂] that rises gradually over the expiratory phase and terminates in a maximum [CO₂] corresponding to end-tidal CO₂, and in which the etCO₂ is the same for every breath. In practice, it will be understood that the capnogram 40 for a real patient usually deviates significantly from this idealized curve due to numerous factors such as non-uniform breathing, talking, coughing, possible chronic lung problems in the case of an ill patient, or so forth. In the capnogram of a real patient, the etCO₂ may vary from breath to breath. The illustrative idealized example of FIG. 2 further assumes a constant respiration rate of 4 seconds/breath, i.e. 15 breaths per minute. As is known in the art, the resting respiration rate (RR) for a normal adult patient is typically on the order of 3-5 seconds/breath (12-20 breaths per minute), with higher RR typically observed for infants (up to about 60 breaths per minute). In a real patient, the RR is generally not constant—the RR can increase significantly due to excitement or exertion, may slow during rest periods, may stop entirely during a sleep apnea episode, and/or may generally vary significantly due to various respiratory ailments or other medical conditions.

With continuing reference to FIGS. 1 and 2, in an operation 42 at a current time t the maximum CO₂ value over a (past) time window W of duration T_{W} is determined. The duration T_{W} of the time window W for the operation 42 is chosen to encompass several breaths. For example, in some embodiments T_{W} has a duration of at least 30 seconds (encompassing five breaths for a patient breathing at a slow RR of 10 breaths/minute, i.e. 6 sec/breath), although shorter values are contemplated, such as for infants whose RR is higher. In some embodiments T_{W} is in the range 30 seconds to 3 minutes inclusive. For an adult, T_{W} may be chosen to be in the range 1 minute to 2 minutes inclusive. Setting T_{W} longer than these illustrative upper limit values is also contemplated, and may be appropriate for example in conjunction with patients who are active or otherwise exhibit significant breath-to-breath variation in the capnogram 40.

The time window W is a sliding time window. That is, the operation 42 determining the largest [CO₂] value in the time window W is repeated (as indicated by repeat operation 44 of FIG. 1) for successive current time values t (and corresponding time shifts of the time window T_{W} as diagrammatically shown in FIG. 2) at a sampling interval T_{S} to generate an etCO₂ signal 50. The sampling interval T_{S} for the repetition 44 is typically much larger than the [CO₂] measurement interval employed by the capnograph 10. For example, the [CO₂] output by the measurement cell 20 may be sampled at 10 millisecond time intervals to generate the capnogram 40, while the sampling interval T_{S} is 10 seconds in illustrative FIG. 2. On the other hand, the sampling interval T_{S} determines the temporal resolution of the etCO₂ signal 50, and so it is preferably chosen to be relatively short, and in particular is much shorter than the duration T_{W} of the sliding time window W. In some embodiments, the sampling interval T_{S} is in the range 5 seconds to 15 seconds inclusive, although longer or shorter sampling intervals are contemplated.

The loop 42, 44 thus implements a sliding window maximum operation 42, 44 in which, for each current time t at which an end-tidal CO₂ sample is taken, the largest [CO₂] value of the capnogram 40 within the time window W(t) is chosen as the etCO₂ value for current time t. The output is the etCO₂ signal 50 which has the advantages (compared with end-tidal CO₂ calculated on a per-breath basis) of being both smoother and a closer approximation of the maximum alveolar CO₂ partial pressure. Another advantage of the etCO₂ signal 50 is that the etCO₂ samples are equally-spaced at the sampling interval T_{S}; whereas, a per-breath end-tidal CO₂ signal is unequally spaced in accord with the breathing intervals (although the per-breath signal can be re-sampled or otherwise post-processed to provide equally-spaced data).

This sliding window maximum processing can be represented mathematically as follows:

etCO₂(t)=max([CO₂])|_{W(t)} (1)

where t denotes time, [CO₂] denotes the capnograph signal 40, the window W(t) is the following portion of the capnogram 40:

W(t)={[CO₂]_{t-T}_{_{W}}, . . . ,[CO₂]ₜ₋₁} (2)

and the function max([CO₂])|_{W(t)} returns the maximum carbon dioxide level over the window W(t). The etCO₂(t) calculation of Expression (1) is repeated at the sampling interval T_{S}, e.g. at times tₒ, tₒ+T_{S}, tₒ+2T_{S}, tₒ+3T_{S}, . . . using corresponding time windows W(tₒ), W(tₒ+T_{S}), W(tₒ+2T_{S}), W(tₒ+3T_{S}), . . . as shown in FIG. 2 to generate the etCO₂ signal 50 as a function of time with sampling interval T_{S}.

As further indicated in FIG. 2, it will be appreciated that the first iteration of this sliding window maximum operation is delayed by a delay time T_{delay}=T_{W} in order to generate the initial window Wₒ. If this delay is considered too long, it is contemplated to use a shorter time window for the first iteration to acquire the first sample of the etCO₂ signal 50 more quickly, albeit with possibly greater error due to the smaller initial window duration.

In Expression (2), the window W(t) is defined to have its right (i.e. highest time value) edge one sample behind the current time t, but more generally a delay D may optionally be employed, that is, more generally:

W(t)={[CO₂]_{t-D-T}_{_{W}}, . . . ,[CO₂]_{t-D}} (2a)

In the window W(t) of Expression (2a), the delay D=0 is a contemplated possibility, and may be used if a stable value for [CO₂]ₜ is available at the time operation 42 is performed.

As noted, the etCO₂ signal 50 is smoothed as compared to the compared with end-tidal CO₂ calculated on a per-breath basis due to smoothing action of taking the maximum value over the time window W. However, any random noise causing an erroneously high CO₂ value will be captured by the sliding window maximum operation 42, 44. In the illustrative embodiment of FIG. 1, this is suppressed by an optional smoothing filter 52, such as a low-pass filter, a digital mean filter, a median filter, or so forth, in order to produce a smoothed etCO₂ signal 54. (Note that the smoothing operation 54 is not depicted in FIG. 2). Additionally or alternatively, suppression of an occasional spuriously high CO₂ value can be suppressed by detailed construction of the max(⋅ ⋅ ⋅ ) operation of Expression (1). For example, the max(⋅ ⋅ ⋅ ) operation may output the second- or third-highest CO₂ value in the window W, or may output the average of the N highest [CO₂] values in the window W (where N is a low positive integer, e.g. N≤4). As yet another approach, a weak smoothing filter (not shown) may be applied to the capnograph signal 40 before applying the operation 42. For example, this weak smoothing filter may be a moving average filter that makes the replacement [CO₂]ₙ←avg{[CO₂]ₙ₋₁, [CO₂]ₙ, [CO₂]ₙ₊₁}.

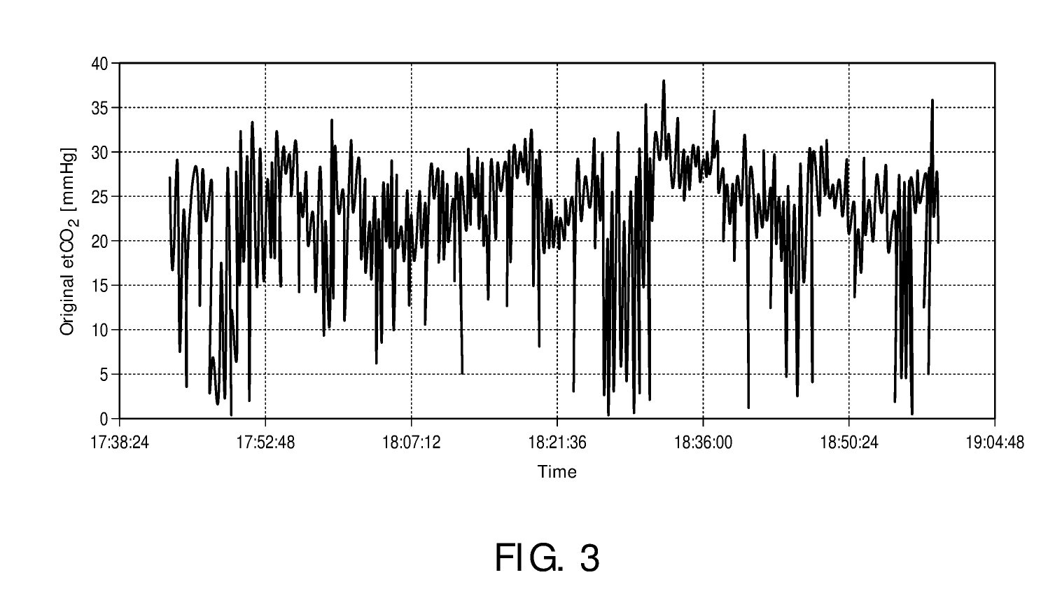

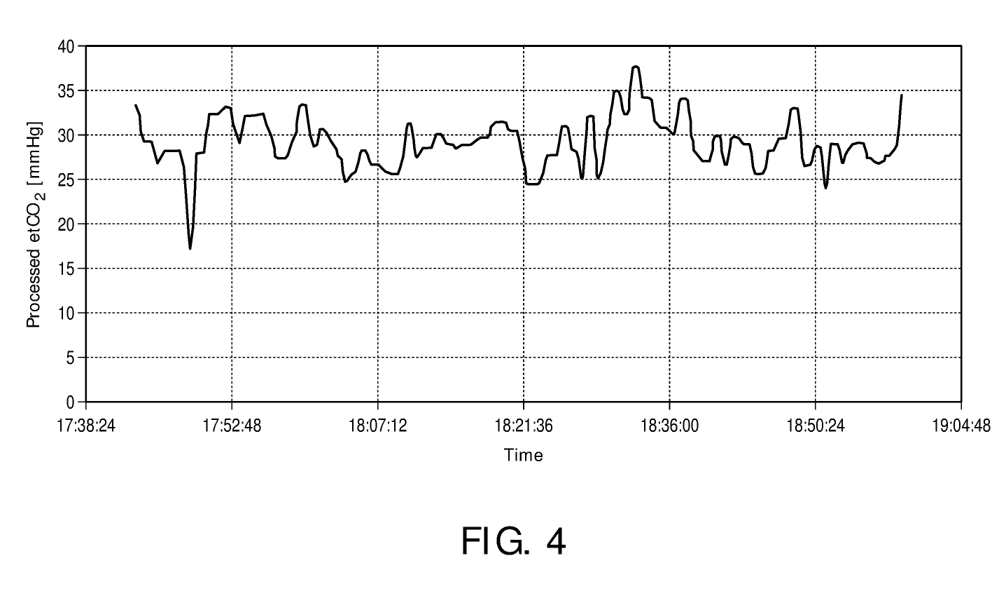

With reference to FIGS. 3 and 4, an illustrative example of the processing loop 42, 44 is shown. FIG. 3 illustrates an experimental example of end-tidal CO₂ measured conventionally by taking the maximum [CO₂] value over each breath. A large amount of “noise” is observed, but it will be noted that the larger-magnitude deviations making up this “noise” are mostly in the downward direction, that is, toward lower [CO₂] value. This reflects the observation made herein that most clinical or physiological sources of error in end-tidal CO₂ (e.g. incomplete flushing of airway dead volume between breaths, parallel alveolar dead volume, impact of supplemental oxygen) tend to reduce the end-tidal CO₂ value produced by capnography on a per-breath basis. That is, the observed deviations are characteristic of systematic error that systematically decreases the end-tidal CO₂ value calculated on a per-breath basis, rather than being characteristic of true random noise.

By contrast, FIG. 4 illustrates the etCO₂ signal 50 produced by applying the sliding window maximum operation 42, 44 to the same capnogram signal that was conventionally processed to produce the end-tidal CO₂ signal of FIG. 3. It is seen that this experimental example of the etCO₂ signal 50 is much less “noisy” in that the predominantly downward deviations are removed, and the etCO₂ value is higher overall than the per-breath end-tidal CO₂ signal of FIG. 3. The etCO₂ signal 50 produced by the sliding window maximum operation 42, 44 is thus a better surrogate for the alveolar maximum CO₂ partial pressure as compared with the end-tidal CO₂ data of FIG. 3.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A capnograph device comprising:
a carbon dioxide measurement component configured to measure respiratory carbon dioxide level; and
an electronic processor programmed to:
generate a capnogram signal comprising respiratory carbon dioxide level measured by the carbon dioxide measurement component as a function of time; and
compute an end-tidal carbon dioxide (etCO₂) signal as a function of time by operations including performing a sliding window maximum operation on the capnograph signal wherein the sliding window maximum operation employs a sliding time window (W) encompassing several breaths.

2. The capnograph device of claim 1 wherein the sliding window maximum operation employs the sliding time window (W) whose duration (T_{W}) encompasses at least five breaths.

3. The capnograph device of claim 1 wherein the sliding window maximum operation employs the sliding time window (W) whose duration (T_{W}) is at least 30 seconds.

4. The capnograph device of claim 1 wherein the sliding window maximum operation employs the sliding time window (W) whose duration (T_{W}) is between one minute and two minutes inclusive.

5. The capnograph device of claim 1 wherein the sliding window maximum operation employs a sampling interval (T_{S}) of between five seconds and fifteen seconds inclusive.

6. The capnograph device of claim 1 wherein the sliding window maximum operation comprises computing the etCO₂ signal as:
etCO₂(t)=max([CO₂])|_{W(t)}
where t denotes time, [CO₂] denotes the capnogram signal, and W(t) denotes the sliding time window (W) as:
W(t)={[CO₂]_{t-D-T}_{_{w}}, . . . ,[CO₂]_{t-D}}
where D is a delay value and D≥0.

7. The capnograph device of claim 1 wherein performing the sliding window maximum operation comprises computing etCO₂(t)=max([CO₂])|_{W(t)} where t denotes time, [CO₂] is the capnogram signal and W(t) is a sliding time window.

8. The capnograph device of claim 1 wherein the electronic processor is programmed to compute the etCO₂ signal as a function of time by operations further including applying a smoothing filter to the capnograph signal prior to performing the sliding window maximum operation on the capnograph signal.

9. The capnograph device of claim 1 wherein performing the sliding window maximum operation computes an unsmoothed etCO₂ signal and the electronic processor programmed to compute a smoothed etCO₂ signal as a function of time by applying a smoothing filter to the unsmoothed etCO₂ signal.

10. The capnograph device of claim 1 further comprising:
a display component configured to display the etCO₂ signal.

11. The capnograph device of claim 1 comprising a sidestream capnograph device including, as a unit, the carbon dioxide measurement component, the electronic processor, and a pump connected to draw respired air though the carbon dioxide measurement component.

12. A non-transitory storage medium storing instructions readable and executable by an electronic processor to perform a capnography method comprising:
generating a capnogram signal comprising respiratory carbon dioxide level measured by a carbon dioxide measurement component as a function of time; and
performing a sliding window maximum operation on the capnograph signal to compute an end-tidal carbon dioxide (etCO₂) signal as a function of time wherein the sliding window maximum operation employs a sliding time window (W) that encompasses several breaths.

13. The non-transitory storage medium of claim 12 wherein the sliding window maximum operation employs the sliding time window (W) whose duration (T_{W}) is at least 30 seconds.

14. The non-transitory storage medium of claim 12 wherein the sliding window maximum operation employs the sliding time window (W) whose duration (T_{W}) is at least one minute.

15. The non-transitory storage medium of claim 12 wherein the sliding window maximum operation employs a sampling interval (T_{S}) of between five seconds and fifteen seconds inclusive.

16. The non-transitory storage medium of claim 12 wherein performing the sliding window maximum operation to compute the etCO₂ signal as a function of time comprises computing etCO₂(t)=max([CO₂])|_{W(t)} where t denotes time, [CO₂] is the capnogram signal and W(t) is a sliding time window.

17. The non-transitory storage medium of claim 16 wherein max([CO₂])|_{W(t)} returns the maximum [CO₂] value over the time window W(t) defined as one of:
(i) the largest capnogram signal sample over the time window W(t);
(ii) the second-largest capnogram signal sample over the time window W(t);
(iii) the third-largest capnogram signal sample over the time window W(t); and
(iv) the average of N highest signal sample over the time window W(t) where N is a positive integer less than or equal to four.

18. The non-transitory storage medium of claim 12 wherein the capnography method further comprises:
applying a smoothing filter to the capnograph signal prior to performing the sliding window maximum operation on the capnograph signal.

19. The non-transitory storage medium of claim 12 wherein the capnography method further comprises:
applying a smoothing filter to the etCO₂ signal to compute a smoothed etCO₂ signal.

20. A capnograph device comprising:
a carbon dioxide measurement component configured to measure respiratory carbon dioxide level; and
an electronic processor (30) as set forth in claim 12;
wherein the capnograph device is one of:
(1) a sidestream capnograph device including, as a unit, the carbon dioxide measurement component, the electronic processor, and a pump connected to draw respired air though the carbon dioxide measurement component; and
(2) a mainstream capnograph device.
